(19) United States
(12) Patent Application Publication
(10) Pub. No.: US 2026/0057993 A1
(43) Pub. Date: Feb. 26, 2026

(54) SYSTEMS AND METHODS FOR REGIMEN ADHERENCE EVALUATION

(71) Applicant: Novo Nordisk A/S, Bagsvaerd (DK)

(72) Inventors: Tinna Bjoerk Aradottir, Copenhagen (DK); Thomas Emil Ryde, Koebenhavn K (DK); Nicholas William Ciccone, Koebenhavn OE (DK); Maria Sejersen Jensen, Aalsgaarde (DK); Henrik Bengtsson, Taastrup (DK)

(21) Appl. No.: 18/874,891

(22) PCT Filed: Jun. 21, 2023
(86) PCT No.: PCT/EP2023/066766

§ 371 Date: Dec. 13, 2024

(30) Foreign Application Priority Data
Jun. 24, 2022 (EP) 22180912.2

(51) Int. Cl. G16H 20/17

(52) U.S. Cl. CPC G16H 20/17

(57) ABSTRACT

A diabetes management system adapted to determine adherence for a subject in treatment according to a basal insulin regimen, the system being adapted to receive regimen data. BG data and insulin injection data. If one or more insulin injections have not been received in accordance with the prescribed regimen and thus are missing, the system is adapted to calculate for each missing injection an expected dose response BG value. By comparing received BG data, corresponding to the missing insulin injections, it can be determined for a given confidence interval whether or not the subject has been in adherence with the basal insulin regimen.

The present disclosure generally relates to systems and methods for assisting patients and health care practitioners in managing insulin treatment to diabetics. In a specific aspect the present invention relates to systems and methods suitable for use in a diabetes management system providing an optimized personalized basal insulin titration regimen.

### BACKGROUND

Diabetes mellitus (DM) is impaired insulin secretion and variable degrees of peripheral insulin resistance leading to hyper-glycaemia. Type 2 diabetes mellitus is characterized by progressive disruption of normal physiologic insulin secretion. In healthy individuals, basal insulin secretion by pancreatic B cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours. Years of poorly controlled hyper-glycaemia can lead to multiple health complications. Diabetes mellitus is one of the major causes of premature morbidity and mortality throughout the world.

Effective control of blood/plasma glucose can prevent or delay many of these complications but may not reverse them once established. Hence, achieving good glycaemic control in efforts to prevent diabetes complications is the primary goal in the treatment of type 1 and type 2 diabetes. Smart titrators with adjustable step size and physiological parameter estimation and pre-defined fasting blood glucose target values have been developed to administer insulin medicament treatment regimens.

There are numerous non-insulin treatment options for diabetes, however, as the disease progresses, the most robust response will usually be with insulin. In particular, since diabetes is associated with progressive β-cell loss many patients, especially those with long-standing disease will eventually need to be transitioned to insulin since the degree of hyperglycemia (e.g., HbA1c>8.5%) makes it unlikely that another drug will be of sufficient benefit.

The ideal insulin regimen aims to mimic the physiological profile of insulin secretion as closely as possible. There are two major components in the insulin profile: a continuous basal secretion and prandial surge after meals. The basal secretion controls overnight and fasting glucose while the prandial surges control postprandial hyperglycemia.

Based on the time of onset and duration of their actions, injectable formulations can be broadly divided into basal (long-acting analogues [e.g., insulin detemir and insulin glargine] and ultra-long-acting analogues [e.g., insulin degludec (for once-daily administration) and insulin icodec (intended for once-weekly administration]) and intermediate-acting insulin [e.g., isophane insulin] and prandial (rapid-acting analogues [e.g., insulin aspart, insulin glulisine and insulin lispro]). Premixed insulin formulations incorporate both basal and prandial insulin components.

Basal insulins will typically be the sole (initial) insulin treatment for type 2 diabetics while for type 1 diabetics a basal insulin can be used in combination with a rapid-acting insulin before meals.

Generally, in order to determine an optimal basal insulin dose for a given patient the patient is titrated starting on an initial safe suggested low dose of basal insulin (typically 10 U/day) which is then increased until Fasting Plasma Glucose (FPG) is within target range, generally 80-130 mg/dL at a dose of typically 40-70 U/day for at type 2 diabetic. Alternatively Fasting Blood Glucose (FBG) values are used. Dose adjustments should be more modest and less frequent as the target comes close and down-titration is recommended in case of occurrence of any hypoglycemia.

However, there are significant barriers not only to initiating treatment with insulin but also to optimizing the dose and intensifying the regimen, all of which are necessary steps to tailor treatment to individual needs and maintain glycaemic control.

One challenge to effective titration is treatment adherence: The failure to initiate, optimize and intensify basal insulin treatment is driven by clinical inertia leading to poor treatment adherence. This typically relates to forgetfulness, perceived need for medication, fear of hypoglycemia, and lack of confidence or uncertainties regarding insulin titration.

Connected injection devices provide insights on treatment adherence and actionable changes to the treatment to patients and clinicians. Some decision support tools, such as insulin titration support applications, use input from connected devices to provide reliable and safe guidance. It is important that the data from the connected device are complete, as the algorithm uses the data to calculate a safe and efficient dose recommendation to the patient. Correspondingly, it is important to know whether a recommended dose has been taken or not.

The nature of injection data is that they are sparse, and they are meant to represent whether the patient is adherent to the treatment regimen or not. Therefore, a “missing” data point represents non-adherence, and thereby an important input to the algorithm.

In the context of bolus calculation the issue of missing dose data has been addressed e.g. in WO 2021/172628 disclosing that evaluation of BG values may indicate whether a given recommended dose was actually taken or whether a recommended dose was not taken due to not being needed, e.g. an expected meal was skipped. Evaluation of BG values is based on general considerations in respect of a set BG target range.

WO 2022/117713 discloses a data collection device to be used in combination with an insulin infusion pump. The device is adapted to substitute missing data based on BG data and using predictive learning. The missing data may be in respect of non-insulin medication such as pain reliever, allergy medication, or cold medication.

WO 2020/043922 discloses a diabetes management system adapted to determine adherence for a subject in treatment according to a basal insulin regimen. The system is adapted to receive regimen data setting out a current dose size and a prescribed injection periodicity, as well as a plurality of fasting blood glucose (FBG) measurements of the subject taken over a time course thereby establishing a FBG history (FBGH), and a plurality of insulin injection data sets during all or a portion of the time course thereby establishing an insulin injection history (IH). The system comprises one or more processors and a memory, the memory comprising instructions that, when executed by the one or more processors, perform data optimization based on FBGH and IH by handling missing data in which one or more temporal gaps in subject data are interpolated by resampling subject data by a predefined time interval.

Having regard to the above-discussed problem of treatment adherence and thus missing dose data during basal insulin titration, it is an object of the present invention to provide methods and systems allowing for more efficient and safer insulin titration despite missing dose data.

### DISCLOSURE OF THE INVENTION

In the disclosure of the present invention, embodiments and aspects will be described which will address one or more of the above objects or which will address objects apparent from the below disclosure as well as from the description of exemplary embodiments.

In summary, the present invention is based on the realization that a “missing” insulin dose data point may occur due to a technical issue, such as lost communication to the injection device or that the patient has not installed the connected add-on device properly to their injection device. Correspondingly, it would be beneficial for a given titration regimen if it was possible to distinguish between non-adherence and missing data points.

Correspondingly, in a first aspect of the invention a diabetes management system adapted to determine adherence for a subject in treatment according to a basal insulin regimen is provided. The system is adapted to receive regimen data setting out a current dose size and a prescribed injection periodicity, a plurality of fasting blood glucose (FBG) measurements of the subject taken over a time course thereby establishing a FBG history (FBGH), each respective glucose measurement in the plurality of glucose measurements comprising (i) a FBG value, and (ii) a corresponding FBG timestamp, and a plurality of insulin injection data sets during all or a portion of the time course thereby establishing an insulin injection history (IH), each injection data set comprising (i) an injection amount, and (ii) an injection timestamp representing when in the time course the injection occurred. The system comprises one or more processors and a memory, the memory comprising instructions that, when executed by the one or more processors, perform a method comprising the steps of: determining if for a period of time one or more insulin injection data sets have not been received (logged) in accordance with the prescribed injection regimen and thus are missing. In case of missing insulin injection data set(s) the method comprises the steps of: based on received FBGH and IH and using a dose response algorithm, calculating for each missing injection an expected dose response FBG value, and for a given confidence interval, determining whether or not the received FBG values, corresponding to the missing insulin injections, correspond to the calculated dose response FBG values. If the received FBG values correspond to the calculated dose response FBG values, the system determines that the subject has been in adherence with the basal insulin regimen, or if the received FBG values do not correspond to the calculated dose response FBG values, the system determines that the subject has not been in adherence with the basal insulin regimen. The FBG values may be derived from received CGM data.

In this way knowledge about the FBGH and IH can be used to determine whether or not non-logged (i.e. missing) injections were actually taken by a patient following a titration regimen.

In an exemplary embodiment the expected dose response FBG values are based on received FBGH and IH as well as regimen dose data, i.e. for the non-logged injections the calculations are based on regimen data setting out time and dose size for the non-logged injections.

In an exemplary embodiment the diabetes management system is adapted to further provide an insulin dose recommendation for the subject, the method comprising the additional steps of receiving a dose guidance request (DGR), and determining if the subject has been in adherence with the regimen in respect of insulin injections for a predetermined amount of time prior to the DGR being received. If the subject has been in adherence (with or without missing dose data), the system provides an updated dose recommendation based on received FBGH and IH, or if the subject has not been in adherence, the system maintains a current dose recommendation. The predetermined amount of time prior to the DGR being received may be the time since a last previous DGR was made.

In a second aspect of the invention a method for determining adherence for a subject in treatment according to a basal insulin regimen is provided. The method comprises the steps of obtaining regimen data setting out a current dose size and a prescribed injection periodicity, obtaining a plurality of fasting blood glucose (FBG) measurements of the subject taken over a time course thereby establishing a FBG history (FBGH), each respective glucose measurement in the plurality of glucose measurements comprising (i) a FBG value, and (ii) a corresponding FBG timestamp, and obtaining a plurality of insulin injection data sets during all or a portion of the time course thereby establishing an insulin injection history (IH), each injection data set comprising (i) an injection amount, and (ii) an injection timestamp representing when in the time course the injection occurred. The method comprises the further steps of determining if for a period of time one or more insulin injection data sets have not been received in accordance with the prescribed injection regimen and thus are missing. In case of missing insulin injection data set(s) the method comprises the further steps of based on received FBGH and IH and using a dose response algorithm, calculating for each missing injection an expected dose response FBG value, for a given confidence interval, determining whether or not the received FBG values, corresponding to the missing insulin injections, correspond to the calculated dose response FBG values. If the received FBG values correspond to the calculated dose response FBG values, the method determines that the subject has been in adherence with the basal insulin regimen, or if the received FBG values do not correspond to the calculated dose response FBG values, the method determines that the subject has not been in adherence with the basal insulin regimen. The FBG values may be derived from received CGM data.

The expected dose response FBG values may be based on received FBGH and IH as well as regimen dose data.

The method may be further adapted to provide an insulin dose recommendation for the subject, the method comprising the additional steps of receiving a dose guidance request (DGR), determining if the subject has been in adherence with the regimen in respect of insulin injections for a predetermined amount of time prior to the DGR being received, if the subject has been in adherence, providing an updated dose recommendation based on received FBGH and IH, or if the subject has not been in adherence, maintaining a current dose recommendation. The predetermined amount of time prior to the DGR being received may be the time since a last previous DGR was made.

### BRIEF DESCRIPTION OF THE DRAWINGS

In the following embodiments of the invention will be described with reference to the drawings, wherein

FIG. 1 shows for an adherent case obtained FBG (dark gray) and dose data (light gray) received from a patient over a time course,

FIG. 2 shows for the adherent case of FIG. 1 the FBG distribution relative to a confidence interval for a calculated response,

FIG. 3 shows for a non-adherent case obtained FBG (dark gray) and dose data (light gray) received from a patient over a time course,

FIG. 4 shows for the non-adherent case of FIG. 3 the FBG distribution relative to a confidence interval for a calculated response, and

FIG. 5 show non-symmetric weighting functions.

### DESCRIPTION OF EXEMPLARY EMBODIMENTS

Overall a diabetes dose guidance system is provided that helps people with diabetes by generating recommended insulin doses. In such a system a given algorithm is used to generate recommended insulin doses and treatment advice for diabetes patients based on BG and insulin dosing history.

Essentially such a system comprises a back-end engine (“the engine”) which is the main aspect of the present invention used in combination with an interacting system in the form of a client and an operating system.

The client from the engine's perspective is the software component that requests dose guidance. The client gathers the necessary data (e.g. CGM data, insulin dose data, patient parameters) and requests dose guidance from the engine. The client then receives the response from the engine.

On a small local scale the engine may run directly as an app on a given user's smartphone and thus be a self-contained application comprising both the client and the engine. Alternatively, the system setup may be designed to be implemented as a back-end engine adapted to be used as part of a cloud-based large-scale diabetes management system. Such a cloud-based system would allow the engine to always be up-to-date (in contrast to app-based systems running entirely on e.g. the patient's smartphone), would allow advanced methods such as machine learning and artificial intelligence to be implemented, and would allow data to be used in combination with other services in a greater “digital health” set-up. Such a cloud-based system ideally would handle a large amount of patient requests for dose recommendations.

Although a “complete” engine may be designed to be responsible for all computing aspects, it may be desirable to divide the engine into a local and a cloud version to allow the patient-near day-to-day part of the dose guidance system to run independently of any reliance upon cloud computing. For example, when the user via the client app makes a request for dose guidance the request is transmitted to the cloud engine which will return a dose recommendation. In case cloud access is not available the client app would run a dose-recommendation calculation using the current local algorithm. Dependent upon the user's app-settings the user may or may not be informed.

In order to provide a safe and efficient dose guidance recommendation it is important that the data on which the recommendation calculations is based is as complete as possible.

Correspondingly, in a first aspect the present invention provides a diabetes management system adapted to determine adherence for a subject in treatment according to a basal insulin regimen.

More specifically, the invention aims to distinguish between periods of non-adherence and periods of missing data in a time series of paired injection and glucose data. This is done by constructing a model of the dose response using periods where adherence is known (i.e. where data points are available) and calculating the probability of a new missing injection data point being due to non-adherence or due to a truly missing injection data point, from looking at the corresponding glucose data.

An advantage of this solution is that it enables a more stable and accurate dose guidance in a setup where insulin pen connectivity is used. By calculating the probability of adherence during periods of missing data from the connected pen, dose guidance which otherwise would have been delayed can be provided as soon as connectivity is restored.

More specifically, an exemplary diabetes management system adapted to determine adherence for a subject in treatment according to a basal insulin regimen is provided. The system is adapted to receive (a) regimen data setting out a current dose size and a prescribed injection periodicity, (b) a plurality of fasting blood glucose (FBG) measurements of the subject taken over a time course thereby establishing a FBG history (FBGH), each respective glucose measurement in the plurality of glucose measurements comprising (i) a FBG value, and (ii) a corresponding FBG timestamp, and (c) a plurality of insulin injection data sets during all or a portion of the time course thereby establishing an insulin injection history (IH), each injection data set comprising (i) an injection amount, and (ii) an injection timestamp representing when in the time course the injection occurred.

The system comprises one or more processors and a memory storing instructions that, when executed by the one or more processors, perform a method comprising the steps of (A) determining that for a period of time one or more insulin injection data sets have not been received in accordance with the prescribed injection regimen and thus are missing, and (B) based on received FBGH and IH and using a dose response algorithm, calculating for each missing injection an expected dose response FBG value.

The method comprises the further steps of (C) for a given confidence interval, determining whether or not the received FBG values, corresponding to the missing insulin injections, correspond to the calculated dose response FBG values, and (i) if the received FBG values correspond to the calculated dose response FBG values, determining that the subject has been in adherence with the basal insulin regimen, or (ii) if the received FBG values do not correspond to the calculated dose response FBG values, determining that the subject has not been in adherence with the basal insulin regimen.

In an exemplary embodiment a patient is using a continuous glucose monitor (CGM) providing BG data based on which fasting BG (FBG) values are determined as well as a connected drug delivery device, e.g. a pen device with an add-on device to log injections in a smartphone app that provides daily insulin titration guidance. The app titrates up by 4 units if the fasting glucose is above range, titrates down by 4 units if the FG is below range, otherwise no change is recommended. This is done every three days.

### Example 1: Adherence

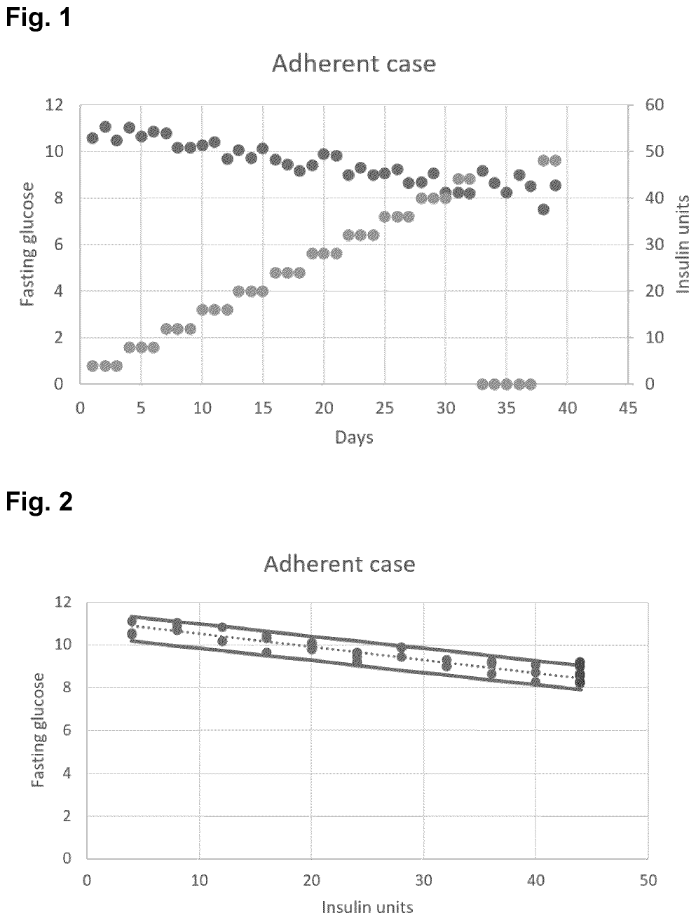

A patient has been adherent (as seen from the logged data) for the first 29 days of the period, and is now taking 40 units (IU) of insulin:
On days 33-37 no injections are logged (see FIG. 1)
On day 38 the patient requests a dose recommendation
A dose response algorithm (see below) creates a dose response for the missing days from the data from days 1-37 assuming that on days 33-37 the dose of 44 units were taken (see FIG. 2)
An evaluation algorithm (see below) identifies that the average of the fasting glucose measurements during days 33-37 falls within the 95% confidence interval of the response
Based on the above the evaluation algorithm determines that the patient has been adherent to taking the 44 units of insulin each day and continues the titration from day 32 by suggesting a dose increase by 4 units

The above is summarized in table 1 below

TABLE 1
Day for day FBG values for a patient in adherence

| Day | FBG | | Insulin |
|---|---|---|---|
| 1 | 10.1789 | 10 | 4 |
| 2 | 10.82865 | 10 | 4 |
| 3 | 10.20576 | 10 | 4 |
| 4 | 10.23753 | 10 | 8 |
| 5 | 10.18877 | 9.8 | 8 |
| 6 | 9.949167 | 9.8 | 8 |
| 7 | 10.36687 | 9.8 | 12 |
| 8 | 10.66409 | 9.6 | 12 |
| . . . | . . . | . . . | . . . |
| 29 | 8.513334 | 8.2 | 40 |
| 30 | 9.288598 | 8.2 | 40 |
| 31 | 8.254615 | 8.2 | 44 |
| 32 | 9.019671 | 8 | 44 |
| 33 | 8.019158 | 8 | — |
| 34 | 8.863158 | 8 | — |
| 35 | 8.75782 | 8 | — |
| 36 | 9.130789 | 8 | — |
| 37 | 8.872677 | 8 | — |
| 38 | 7.940873 | 7.5 | 48 |
| 39 | 8.653501 | 7.5 | 48 |

### Example 2: Non-adherence

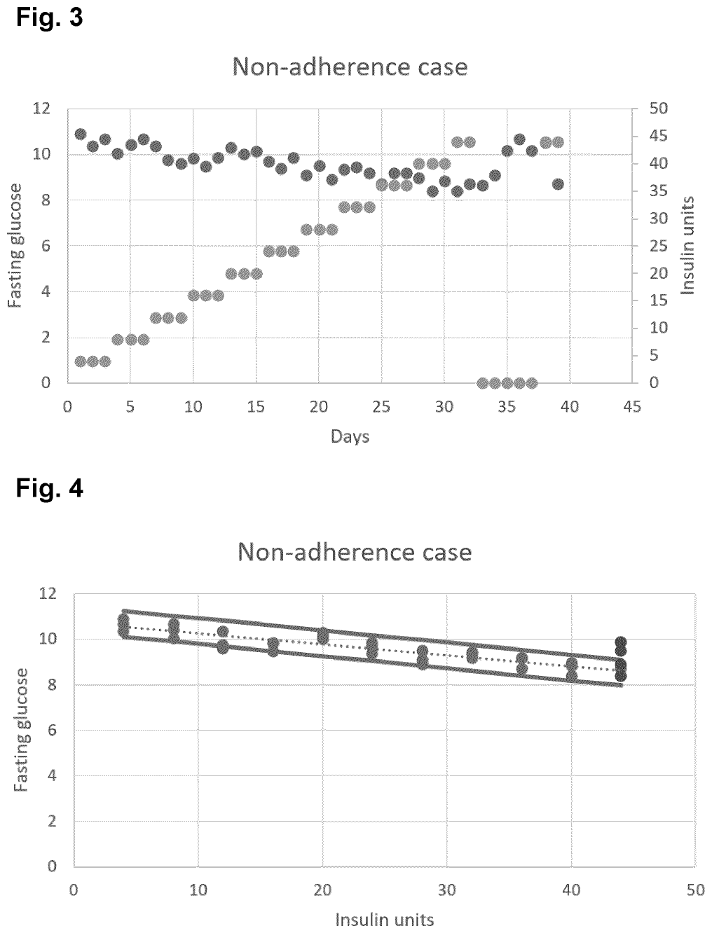

A patient has been adherent (as seen from the logged data) for the first 29 days of the period, and is now taking 40 units of insulin:
On days 33-37 no injections are logged (see FIG. 3)
On day 38 the patient requests a dose recommendation
The dose response algorithm calculates a dose response from the data from days 1-37 assuming that on days 33-37 the dose of 44 units were taken (see FIG. 2)
The evaluation algorithm identifies that the average of the fasting glucose measurements during days 33-37 falls outside of the 95% confidence interval of the response
Based on the above the evaluation algorithm determines that the patient has been non-adherent and continues the titration from day 32 by suggesting no dose change

TABLE 2
Day for day FBG values for a patient in non-adherence

| Day | FG | Insulin |
|---|---|---|
| 1 | 10.88479 | 4 |
| 2 | 10.34878 | 4 |
| 3 | 10.67424 | 4 |
| 4 | 10.0605 | 8 |
| 5 | 10.4195 | 8 |
| 6 | 10.68913 | 8 |
| 7 | 10.34061 | 12 |
| 8 | 9.776278 | 12 |
| . . . | . . . | . . . |
| 29 | 8.382574 | 40 |
| 30 | 8.838843 | 40 |
| 31 | 8.399864 | 44 |
| 32 | 8.724705 | 44 |
| 33 | 8.630002 | — |
| 34 | 9.123819 | — |
| 35 | 10.16755 | — |
| 36 | 10.68806 | — |
| 37 | 10.19095 | — |
| 38 | 10.51138 | 44 |
| 39 | 8.740865 | 44 |

In examples 1 and 2 for a period of 5 injections in accordance with the prescribed regimen all injections are missing. The period may be shorter or longer just as one or some of the injection dose sizes may be known.

In the above examples the dose response algorithm is based on a Global Linear Regression model assuming a linear relationship between glucose measurements, i.e.

$${G}_{i} = α - β {I}_{i} + {e}_{i} , i = 1 , 2 … , k \qquad ( 1 )$$

where Gᵢ represents fasting glucose of day I and Iᵢ is the insulin injection taken the corresponding day.

In the following methods for use in model identification will be described. The methods are used to investigate whether the expected dose response is detectable from the data, and whether outliers in the data have a significant effect on the model identification. Subsequently parameters from the three model structures are identified and it is identified which model gives the best fit to the individual dose response.

To estimate a and B the ordinary least (LSQ) method is used. It can be written for all n points for an individual on matrix form,

$$y = X θ + e \qquad ( 2 )$$

Where y=[G₁, G₂, . . . , Gₙ]^{T}, X=[1, x] with x=[I₁,I₂, . . . , Iₙ]^{T}, e=[e₁, e₂, . . . , eₙ]^{T} and 0=[α, β]^{T}. The ordinary LSQ estimation assumes that the residuals

$$r = y - X \hat{θ} \qquad ( 3 )$$

Where r=[r₁, r₂, . . . , rₙ] are normally distributed around the mean response X \hat{θ}. \hat{θ} is the estimated parameters, and is found by minimizing the sum of squared residuals,

$$\hat{θ} = arg {min}_{θ} {\sum }_{i = 1}^{n} {r}_{i}^{2} = {‖ r ‖}_{2}^{2} = {‖ y - X \hat{θ} ‖}_{2}^{2} \qquad ( 4 )$$

The solution is a vector, \hat{θ}, which is an estimate of the unknown parameters θ. By substituting the expression (3) into the 2-norm we get

$${‖ r ‖}_{2}^{2} = {r}^{T} r = \frac{1}{2} {( y - X \hat{θ} )}^{T} ( y - X \hat{θ} ) {\hat{θ}}^{T} {X}^{T} X \hat{θ} - 2 {\hat{θ}}^{T} {X}^{T} y + {y}^{T} y \qquad ( 5 )$$

This expression with respect to e is minimized and so the derivative with respect to the parameters is taken, which gives the normal equations

$$( {X}^{T} X ) \hat{θ} = {X}^{T} y \qquad ( 6 )$$

And solving for \hat{θ} gives the estimate of the unknown parameters,

$$\hat{θ} = {( {X}^{T} X )}^{- 1} {X}^{T} y \qquad ( 7 )$$

The distribution of the parameter estimate is

$$\hat{θ} ∼ N ( θ , {{σ}^{2} [ {X}^{T} X ]}^{- 1} ) \qquad ( 8 )$$

Where σ² is the estimated noise covariance,

$${σ}^{2} = \frac{1}{n - {n}_{θ}} {\sum }_{i = 1}^{n} {r}_{i}^{2} \qquad ( 9 )$$

And n and n_{θ} are the number of datapoints and parameters, respectively.

The estimated noise covariance is used to calculate the confidence interval, e.g.

$$\hat{y} \pm z \frac{σ}{\sqrt{n}}$$

where ý is the predicted glucose value, z indicates the confidence interval (e.g. 90% vs. 95%) and n is the number of data points.

In the shown examples all data points weigh the same in the ordinary LSQ method, however, these points can be weighed differently, e.g. by the robust LSQ method:

In the ordinary LSQ method, it was assumed that all data points were of equal quality. However, this may not be the case considering the level of variability in the SMBG data. To mini-mize the sensitivity of the fit to outliers and errors, weighted LSW can be used, where instead of minimizing the term in (4), it is minimized

$$\hat{θ} = arg min ( θ ) {\sum }_{i = 1}^{n} {w}_{i}^{2} {r}_{i}^{2} = \frac{1}{2} {‖ r ‖}_{2}^{2} = \frac{1}{2} {‖ W ( y - X \hat{θ} ) ‖}_{2}^{2} \qquad ( 10 )$$

Where wᵢ is the weight of residual rt. The weight can be chosen in different ways using knowledge about the data. The normal equations become

$$( {X}^{T} {W}^{T} WX ) \hat{θ} = {X}^{T} {W}^{T} y \qquad ( 11 )$$

Where W=diag (w₁, w₂, . . . , wₘ) and solving for \hat{θ} gives the estimate of the unknown parameters,

$$\hat{θ} = {( {X}^{T} {W}^{T} WX )}^{- 1} {X}^{T} {W}^{T} y \qquad ( 12 )$$

The distribution of the parameter estimate is then

$$\hat{θ} ∼ N ( θ , {{\hat{σ}}^{2} [ {X}^{T} {W}^{T} WX ]}^{- 1} ) \qquad ( 13 )$$

Here the bi-square weighting is used for robust LSQ, which minimizes the influence of outliers on the fit. The method is iterative and gives full weight to small residuals, and zero weight to residuals larger than expected by random chance. The weights are iteratively calculated by

$${w}_{i} = ( \vert {u}_{i} \vert < 1 ) {( 1 - {u}_{i}^{2} )}^{2} \qquad ( 14 )$$

Where uᵢ is the adjusted and normalized residual rᵢ of the weighted LSQ.

$${u}_{i} = \frac{{r}_{i}}{Ks \sqrt{1 - {h}_{i}}} \qquad ( 15 )$$

Here, hᵢ is the leverage of residuals rᵢ, i.e., the degree by which the i-th residual influences the fit, K is a tuning constant and s is the robust variance,

$$s = \frac{median ( \vert r \vert )}{0.6745} , K = 4.685 \qquad ( 16 )$$

The strength of the bi-square weighting is in its ability to fit the data in a similar manner as the ordinary LSQ method, while eliminating the effect of the outliers.

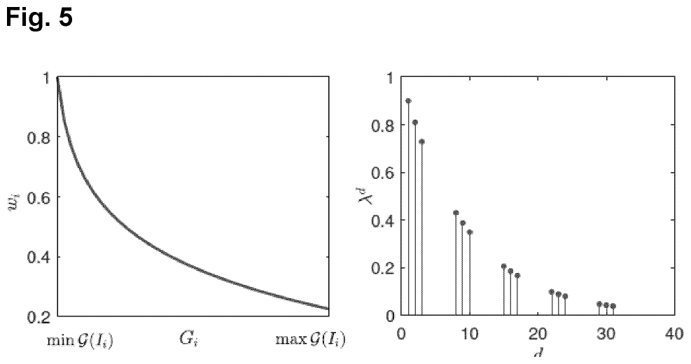

Alternatively, a non-symmetric weighting function considering time or user errors can be used as shown in FIG. 5 which in the left figure shows the non-symmetric weighting function of residuals in (13). The right figure shows the forgetting weights in (14) for an effective memory hori-zon of 10 days. It should be noticed that only three out of seven points are illustrated. This is due to the structure of the SMBG data where only the three last values prior to a dose adjust-ment are available.

If a person measures SMBG at other time points by mistake, the glucose concentration is expected to be equal or hight than the actual pre-breakfast SMBG. This would in general result in an error in the SMBG measurements in the positive direction. It can therefore be expected that outliers caused by user errors would tend to elevate the measured glucose concentration.

Considering the user error and severity of low glucose, a weighting function is designed such that low SMBG values are weighed higher that high SMBG values. However, it should be kept in mind that insulin does affect the glucose levels and it is not desirable to eliminate information about the dose response. SMBG readings are therefore weighed compared to other SMBG readings where the same insulin dose was given. The weighting function is therefore

$${w}_{i} = 1 - \frac{log ( {G}_{i} - min [ G ( {I}_{i} ) ] + 1 )}{log ( max [ G ( {I}_{i} ) ]} \qquad ( 17 )$$

where Gᵢ is the i^{th} SMBG measurement, It is the corresponding insulin injection, and G (Iᵢ) is all SMBG measurements Gᵢ for j=1, 2, . . . , n where Iⱼ=Iᵢ. When there is only one SMBG measurement for the corresponding insulin dose, then dim (G(Iᵢ))=1 and wᵢ=1. The weighting functions is illustrated to the left in FIG. 5.

As indicated above, the determination of adherence or non-adherence can be used to provide an insulin dose recommendation for the subject in an efficient and safe way. More specifically, in the above-described system the performed method could comprise the additional steps of receiving a dose guidance request (DGR) and determining if the subject has been in adherence with the regimen in respect of insulin injections for a predetermined amount of time prior to the DGR being received. If the subject has been in adherence, the system would provide an updated dose recommendation based on received FBGH and IH (e.g. 48 units as described above), or if the subject has not been in adherence, the system would maintain a current dose recommendation (e.g. 44 units as described above).

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A diabetes management system adapted to determine adherence for a subject in treatment according to a basal insulin regimen, the system being adapted to receive:
regimen data setting out a current dose size and a prescribed injection periodicity,
a plurality of fasting blood glucose (FBG) measurements of the subject taken over a time course thereby establishing a FBG history (FBGH), each respective glucose measurement in the plurality of glucose measurements comprising:
(i) a FBG value, and
(ii) a corresponding FBG timestamp,
a plurality of insulin injection data sets during all or a portion of the time course thereby establishing an insulin injection history (IH), each injection data set comprising:
(i) an injection amount, and
(ii) an injection timestamp representing when in the time course the injection occurred,
wherein the system comprises one or more processors and a memory, the memory comprising instructions that, when executed by the one or more processors, perform a method comprising the steps of:
determining if for a period of time one or more insulin injection data sets have not been received in accordance with the prescribed injection regimen and thus are missing, and in case of missing insulin injection data set(s):
based on received FBGH and IH and using a dose response algorithm, calculating for each missing injection an expected dose response FBG value,
for a given confidence interval, determining whether or not the received FBG values, corresponding to the missing insulin injections, correspond to the calculated dose response FBG values, and
if the received FBG values correspond to the calculated dose response FBG values, determining that the subject has been in adherence with the basal insulin regimen, or if the received FBG values do not correspond to the calculated dose response FBG values, determining that the subject has not been in adherence with the basal insulin regimen.

2. A diabetes management system as in claim 1, wherein the expected dose response FBG values are based on received FBGH and IH as well as regimen dose data.

3. A diabetes management system as in claim 1, further adapted to provide an insulin dose recommendation for the subject, the method comprising the additional steps of:
receiving a dose guidance request (DGR),
determining if the subject has been in adherence with the regimen in respect of insulin injections for a predetermined amount of time prior to the DGR being received,
if the subject has been in adherence, providing an updated dose recommendation based on received FBGH and IH, or
if the subject has not been in adherence, maintaining a current dose recommendation.

4. A diabetes management system as in claim 1, wherein the predetermined amount of time prior to the DGR being received is the time since a last previous DGR was made.

5. A diabetes management system as in claim 1, wherein the FBG values are derived from received CGM data.

6. A method for determining adherence for a subject in treatment according to a basal insulin regimen, comprising the steps:
obtaining regimen data setting out a current dose size and a prescribed injection periodicity,
obtaining a plurality of fasting blood glucose (FBG) measurements of the subject taken over a time course thereby establishing a FBG history (FBGH), each respective glucose measurement in the plurality of glucose measurements comprising:
(i) a FBG value, and
(ii) a corresponding FBG timestamp,
obtaining a plurality of insulin injection data sets during all or a portion of the time course thereby establishing an insulin injection history (IH), each injection data set comprising:
(i) an injection amount, and
(ii) an injection timestamp representing when in the time course the injection occurred,
determining if for a period of time one or more insulin injection data sets have not been received in accordance with the prescribed injection regimen and thus are missing, and in case of missing insulin injection data set(s):
based on received FBGH and IH and using a dose response algorithm, calculating for each missing injection an expected dose response FBG value,
for a given confidence interval, determining whether or not the received FBG values, corresponding to the missing insulin injections, correspond to the calculated dose response FBG values, and
if the received FBG values correspond to the calculated dose response FBG values, determining that the subject has been in adherence with the basal insulin regimen, or
if the received FBG values do not correspond to the calculated dose response FBG values, determining that the subject has not been in adherence with the basal insulin regimen.

7. A method as in claim 6, wherein the expected dose response FBG values are based on received FBGH and IH as well as regimen dose data.

8. A method as in claim 6, further adapted to provide an insulin dose recommendation for the subject, the method comprising the additional steps of:
receiving a dose guidance request (DGR),
determining if the subject has been in adherence with the regimen in respect of insulin injections for a predetermined amount of time prior to the DGR being received,
if the subject has been in adherence, providing an updated dose recommendation based on received FBGH and IH, or
if the subject has not been in adherence, maintaining a current dose recommendation.

9. A method as in claim 6, wherein the predetermined amount of time prior to the DGR being received is the time since a last previous DGR was made.

10. A method as in claim 6, wherein the FBG values are derived from received CGM data.
